(19) United States
(12) Patent Application Publication
(10) Pub. No.: US 2023/0263962 A1
(43) Pub. Date: Aug. 24, 2023

(54) SYRINGE-SUPPORT APPARATUS AND METHODS OF USE

(71) Applicants: David F DALURY, Reisterstown, MD (US); Brooke ANDERSON, New York, NY (US)

(72) Inventors: David F Dalury, Reisterstown, MD (US); Brooke Anderson, New York, NY (US); Christopher Dykes, Punka Gorda, FL (US)

(21) Appl. No.: 18/017,370

(22) PCT Filed: Nov. 18, 2020
(86) PCT No.: PCT/US2020/061043

§ 371 Date: Jan. 20, 2023

Related U.S. Application Data
(60) Provisional application No. 63/071,427, filed on Aug. 28, 2020.

(51) Int. Cl. A61M 5/315; A61M 5/48

(52) U.S. Cl. CPC A61M 5/3158; A61M 5/482

(57) ABSTRACT

Apparatus and method for syringe support are disclosed. The syringe-support apparatus may include a front arm and a back arm connected at a hinge mechanism. The front and back arms may comprise a finger grip and thumb grip. A user may employ a well-known scissor-like movement in view of the finger grip and thumb grip, combined with the hinge mechanism. The front and back arms may further comprise a syringe securing mechanism, which may correlate hand motion with a syringe plunger motion. In view of the finger grip, thumb grip, and syringe securing mechanism, respectively, a user may engage the user's forearm muscles during aspiration and injection. The securing mechanism may comprise an adjustable barrel support and barrel support hook to secure a syringe barrel.

### BACKGROUND OF THE INVENTION

Health professionals, as well as allied health professionals, may administer about 16 billion medicine injections worldwide. To the person receiving the injection, the quality of the injection may correlate directly to the level of pain relief In contrast, the person delivering the injection may evaluate the quality of the injection in view of injection tool characteristics, such as ease of use and accuracy, as well as comfort. Accordingly, those who administer injections evaluate the quality of the injection delivery.

An individual health professional may administer multiple injections in any given day. As with all motions, repetition may lead to muscle fatigue and pressure points. In the case of injections, health professionals may experience fatigue in their hands, specifically in their thumbs and the basal joint from the repetitive motion. Further, health professionals may experience fatigue in their hands from the pressure of the syringe injection, itself. Accordingly, health professionals and allied health professionals may experience muscle fatigue and discomfort in their hands when administering injections.

Health and allied health professionals may question the accuracy of an administered injection; specifically, the accuracy of the tool administering the injection. Accuracy, for example, may represent how much control, such as volume, speed, and location of injection, a health professional has over administration of the injection. Accordingly, a health professional administering an injection considers the tool used to deliver the injection when evaluating accuracy of the injection.

### SUMMARY OF THE PRESENT INVENTION

Various exemplary embodiments of the present disclosure may demonstrate one or more of the invention features. Other features and advantages of this invention will become apparent from the following detailed description of the presently preferred embodiments of the invention, taken in conjunction with the accompanying drawings.

In accordance with an exemplary embodiment, a syringe-support apparatus includes a front arm comprising a finger grip, front mount, and a barrel support, and a back arm comprising a thumb grip, a back mount, and a plunger flange support. The syringe-support apparatus further includes a hinge mechanism to move the front arm toward or away from the thumb grip. A syringe-support apparatus further includes a compressible barrel support and barrel flange hatch to secure a barrel to the syringe-support apparatus. The syringe-support apparatus further includes a rotatable plunger flange support and pocket.

In accordance with another exemplary embodiment, a method of seating a syringe in a syringe-support apparatus includes positioning a sized barrel support in a front mount of a front arm. A method of seating a syringe in a syringe-support apparatus further includes engaging a hinge mechanism to receive a syringe barrel in the sized barrel support and contracting the sized barrel support onto a syringe barrel. A method of seating a syringe in a syringe-support apparatus further includes receiving and securing a syringe plunger flange in a plunger support mechanism.

It is to be understood that both the foregoing general description and the following detailed description are exemplary and explanatory only and are not restrictive of the present disclosure or claims.

### BRIEF DESCRIPTION OF THE DRAWINGS

The drawings referenced herein are incorporated in and form part of the specification. The drawings illustrate one or more exemplary embodiments of the present disclosure and together with the description serve to explain various principles and operations. Implications that the drawings illustrate all embodiments of the invention are not to be made.

FIG. 1 illustrates a top-front perspective view of an exemplary embodiment of a syringe-support apparatus in accordance with the present disclosure.

FIG. 2 illustrates a top-front perspective view of an exemplary embodiment of a hinge mechanism of a syringe-support apparatus in accordance with the present disclosure.

FIG. 3A illustrates a side view of an exemplary embodiment of a syringe-support apparatus with open arms in accordance with the present disclosure.

FIG. 3B illustrates a side view of an exemplary embodiment of a syringe-support apparatus with closed arms in accordance with the present disclosure.

FIG. 4 illustrates a side view of an exemplary embodiment of front arm of a syringe-support apparatus in accordance with the present disclosure.

FIG. 5 illustrates an exploded, top perspective view of an exemplary embodiment of syringe securing mechanism of a syringe-support apparatus in accordance with the present disclosure

FIG. 6 illustrates an exploded, top-back perspective view of an exemplary embodiment of a hollow-shaped barrel flange securing mechanism of a syringe support apparatus in accordance with the present disclosure.

FIG. 7A illustrates an exploded, top-back perspective view of an exemplary embodiment of a saddle-shaped barrel flange securing mechanism of a syringe support apparatus in accordance with the present disclosure.

FIG. 7B illustrates an exploded, back-front perspective view of an exemplary embodiment of a saddle-shaped barrel flange securing mechanism of a syringe support apparatus in accordance with the present invention.

FIG. 8 illustrates a flow diagram showing seating a syringe in a syringe-support apparatus in accordance with the present disclosure.

### DETAILED DESCRIPTION OF THE INVENTION

Reference will now be made in detail to various exemplary embodiments of the present disclosure, examples of which are illustrated in the accompanying drawings.

It will be readily understood that the components of the present invention, as generally described and illustrated in the figures herein, may be arranged and designed in a wide variety of different configurations. Thus, the following detailed description of the embodiments of the syringe-support apparatus and method of use of the present invention, as presented in the figures, is not intended to limit the scope of the invention, as claimed, but is merely representative of selected embodiments of the invention.

Reference throughout this specification to “a select embodiment,” “one embodiment,” or “an embodiment” means that a particular feature, structure, or characteristic described in connection with the embodiment is included in at least one embodiment of the present invention. Thus, appearance of the phrases “a select embodiment,” “in one embodiment,” “an exemplary embodiment” or “in an embodiment” in various places throughout this specification are not necessarily referring to the same embodiment.

Features, structure, or characteristics described herein may be combined in any suitable manner in one or more embodiments. One skilled in the relevant art will recognize, however, that the invention may be practiced without one or more of the specific details, or with other methods, components, or materials. In other instances, well-known materials or processes are not shown or described in detail to avoid obscuring aspects of the invention. The following description, which shows by way of illustration a specific embodiment in which the invention may be practiced, is intended only by way of example. That is, the following description simply illustrates certain selected embodiments of a syringe-support apparatus and methods of use that are consistent with the invention as claimed herein. It is to be understood that other embodiments may be utilized because structural and process changes may be made without departing from the scope of the present invention.

Health and allied health professionals, hereinafter referred to collectively as “users,” may commonly use syringes to deliver medicine and other fluids and semi-solids to patients. A syringe may generally comprise a barrel and a plunger. A barrel generally may comprise a barrel flange at one end and a needle lock or opening at an opposite end. A plunger may comprise a plunger flange on one end and seal material on an opposite end. In general, a user may push on a plunger flange to move the seal end of a plunger. The plunger may slide horizontally through a barrel and toward a needle lock, until a barrel flange stops further movement of the plunger. The needle lock may generally secure a needle to the barrel. Put together, a sliding plunger may be pushed or pulled inside a precise-sized barrel to draw in or expel an amount of fluid through a needle. Accordingly, a syringe may function to move fluid within a barrel.

A user may use a syringe to perform two key functions: (1) collecting medicine for delivery to a patient by drawing the medicine into the barrel; and (2) delivering the medicine to the patient. Syringes may perform these functions through aspiration and injection, respectively. Aspiration is when a user uses the plunger flange to pull up on a plunger, drawing a substance, such as a medicine, into a barrel through a needle or opening. Injection is when a user presses down on the plunger flange, moving the plunger horizontally forward in the syringe barrel and expelling the substance from the barrel and through the needle or opening. As described above, the plunger flange may come in contact with the barrel flange, stopping further forward motion of the plunger within the barrel. Accordingly, a syringe may perform aspiration and injection functions by moving a plunger horizontally in a barrel.

Users may administer an estimated 16 billion syringe-based injections annually worldwide. To do so, a user may generally hold a syringe with one hand and may need to shift the hand positioning when moving between aspiration and injection. For example, to inject medicine from a syringe barrel and through a syringe needle, a user may use two fingers to steady a syringe barrel by gripping a barrel flange. A user may engage the user's thumb to push a plunger flange in, and, thus, move a plunger within and horizontally along the barrel. As the plunger moves, the user may push medicine through the needle. To aspirate, a user may need to change grips and use two fingers to grasp the plunger flange and pull back on it while holding the syringe barrel steady. Accordingly, syringe users may experience time loss, inefficiency and other procedural inefficiencies between aspiration and injection.

Current syringe design may engage merely user thumb power, so a user's two-finger hold on a syringe raises further concerns. For example, a user may generally apply inconsistent, relatively weak, and potentially inaccurate force while delivering medicine using a syringe. Also, a user may experience hand fatigue, which may impact the user's control over injection placement and injection delivery speed. Any given user may perform multiple aspirations and injections on any given day. Users may experience hand fatigue because injections require a fair amount of force due to volume and viscosity of the fluid delivered via injection. As a user's hand grip weakens, the user may provide less control over injection placement and volume. Further, in many situations, a diameter of a syringe, as well as a needle size of the syringe may dictate resistance force to an injection. A syringe with a large diameter may require a user to impose greater force on a plunger flange than a syringe with a smaller diameter; however, a user may require multiple syringes to deliver a set volume of medicine when choosing a smaller diameter syringe over a larger diameter syringe. A user may also consider that using multiple smaller diameter syringes may increase procedure time and margin for error, such as by mismeasurement or spillage. To that end, a user must decide between hand fatigue and procedural inefficiency. Accordingly, health and allied health professionals may look for improvement in delivering medicine with syringes.

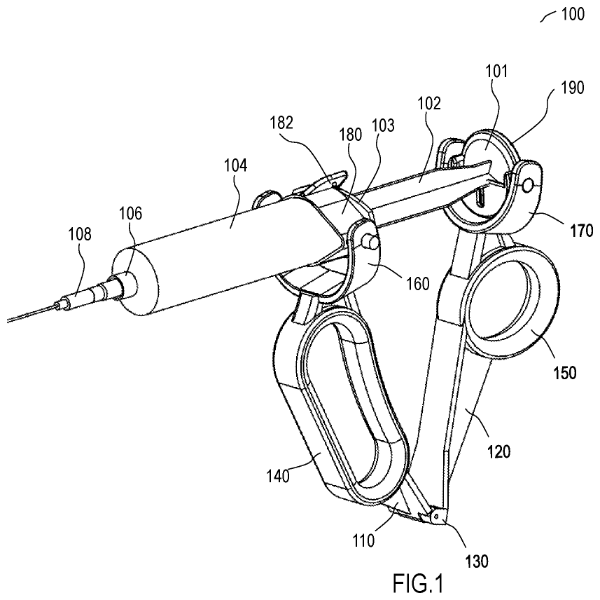

A syringe-support apparatus and method of use of the present invention may address these issues. FIG. 1 illustrates a top-front perspective view of an exemplary embodiment of a syringe-support apparatus (100) in accordance with the present disclosure. A syringe may be secured in a syringe-support apparatus (100) and may comprise a plunger flange (101) a plunger (102), a barrel flange (103) and a barrel (104), with needle (108) in communication with the barrel (104) at a needle lock (106). In an exemplary embodiment, a syringe-support apparatus (100) may comprise a front arm (110), a back arm (120), and a hinge mechanism (130). In an embodiment, for example, the front arm (110) and the back arm (120) may pivotally communicate and lock at the hinge mechanism (130). In an exemplary embodiment, the front arm (110) may comprise a finger grip (140), a front mount (160), a barrel support (180), and a barrel support hook mechanism (182), such as a barrel flange hook. In an exemplary embodiment, the finger grip (140) may comprise molded edges, which may increase comfort. In an embodiment, for example, the barrel support (180) may be of variable sizes. Accordingly, a syringe-support apparatus of the present invention may restrict movement of a syringe barrel and syringe barrel flange.

In an embodiment, for example, the back arm (120) may comprise a thumb grip (150), a back mount (170), and a plunger flange support (190). In an exemplary embodiment, the plunger flange (101) may rotate on an axis, as the plunger flange support (190) may rotate on an axis while securing the plunger flange (101). In an exemplary embodiment, the thumb grip (150), such as a thumb grip loop, may comprise molded edges, which may increase comfort and allow forward and backward motion. Accordingly, a syringe-support apparatus of the present invention may communicate with and secure a syringe.

In an exemplary embodiment, the barrel support (180) may be removable. In an embodiment, the barrel support (180) may comprise flexible material or other material that may conform, at least in part, to a syringe barrel shape. In an embodiment, the barrel support (180) may comprise, for example, a saddle shape, wherein a barrel (104) may snap into the saddle seat. In an alternative embodiment, the barrel support (180) may comprise, for example, a hollow shape, wherein the hollow shape may receive the barrel (104) and may conform to the syringe barrel shape. For example, in an embodiment, some portion of an inner surface of the barrel support (180) may conform to some portion of an outer surface of the barrel support (180). Further, in an exemplary embodiment, the barrel support (180) may comprise a barrel flange hook mechanism, such as a barrel flange hook, to secure the barrel flange (103) and, thus, the syringe barrel (104). Accordingly, a syringe-support apparatus of the present invention may adjust to a syringe barrel shape and size and may restrict movement of a syringe barrel.

With reference to FIG. 1, in an exemplary embodiment, a plunger flange support (190) may secure a plunger flange (101) in communication with a plunger (102). For example, in an embodiment, a plunger flange support (190) may secure a plunger flange (101) by means of a plunger flange hook mechanism, such as a hook, and a plunger flange pocket. In an embodiment, for example, securing the plunger flange (101) to the plunger flange support (190) may couple movement of the plunger flange (101) to movement of the back arm (120). To that end, in an exemplary embodiment, securing the plunger flange (101) to the plunger flange support (190) may couple movement of the plunger (102) to movement of the back arm (120). Accordingly, a syringe-support apparatus of the present invention may couple movement of a syringe plunger to movement of a back arm.

In an exemplary embodiment, a user may engage a hinge mechanism (130) and a thumb grip (150) to move the back arm (120) relative to the front arm (110). In this way, for example, movement of the back arm (120), in an embodiment, may couple to movement of the thumb grip (150). Further, in an exemplary embodiment, because movement of the plunger (102) may couple to movement of the back arm (120), movement of the plunger (102) may couple to movement of the thumb grip (150). In an embodiment, for example, the user may engage the thumb grip (150) and may control a linear speed of the plunger (102) as the plunger (102) travels through the barrel (104). That is, in an exemplary embodiment, a user may propel a plunger (102) forward while engaging a thumb grip (150) and moving the thumb grip (150) forward. Alternatively, in an exemplary embodiment, the user may halt forward movement of the plunger (102) while engaging the thumb grip (150) and halting movement of the thumb grip (150). Further, in an exemplary embodiment, a plunger flange support (190) may be rotatable, which may allow for smoother movement of the plunger (102) through the barrel (104) throughout movement of the plunger. Similar to a removable barrel support (180), which may be secured to the front mount (160) with tabs through front mount (160) openings, the plunger flange support (190) may be removable and secured to the back mount (170) with tabs through back mount (170) openings. Accordingly, a syringe-support apparatus of the present invention may secure a plunger flange and may couple movement of a syringe plunger to movement of a thumb grip.

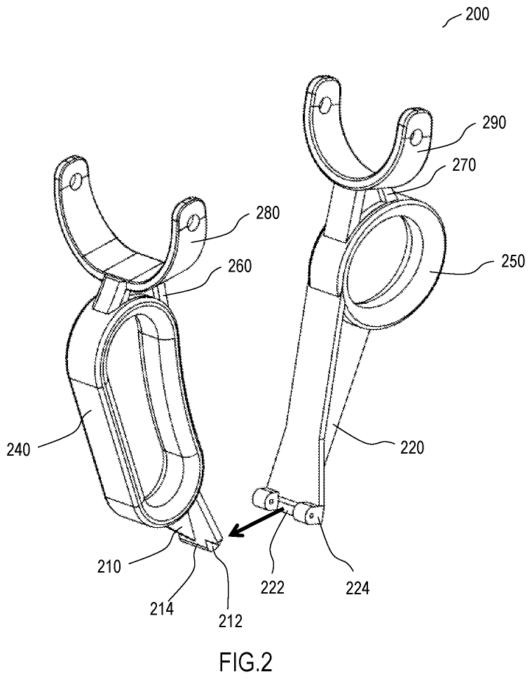

As described herein above, a syringe-support apparatus (100) as described in FIG. 1, may comprise multiple elements, and the multiple elements support use of the syringe-support apparatus (100). In an embodiment, a method of use of a syringe-support apparatus (100) may comprise, for example, and as described herein and below, pivotally associating a front arm (110) to a back arm (120) at a hinge mechanism (130), securing a syringe barrel flange (103) and syringe barrel (104) to barrel support (180) in communication with a front mount (160) of a front arm (110). In an embodiment, a method of use of a syringe-support apparatus (100) may further comprise, for example, securing a plunger flange (101) to a rotatable and, for example, removable, plunger flange support (190) in communication with a back mount (170) of the back arm (120). In an exemplary embodiment, a method of use of a syringe-support apparatus may further comprise engaging the thumb grip (150) and controlling movement of a plunger (102) through a barrel (104). The elements, either alone or in combination, may provide for an improved syringe user's experience, as further described herein below in FIGS. 2-8.

In an exemplary embodiment, a syringe-support apparatus (100), as described in FIG. 1, may control user hand movement with of a hinge mechanism (130). An exemplary embodiment of a syringe-support apparatus (100) may employ a well known scissor-type grip for either aspiration or injection. FIG. 2 illustrates top-front perspective view of an exemplary embodiment of hinge mechanism (200) in accordance with the present disclosure. In an exemplary embodiment, a hinge mechanism (200) may comprise a front arm (210) and a back arm (220). In an embodiment, for example, a front arm (210) may comprise a front hinge element (212) near or at a front arm first end (214) and a front mount (280) at a second end (260). In a further embodiment, for example, a back arm (220) may comprise a back hinge element (222) near or at a back arm first end (224) and a back mount (270) at a second end (290). In an exemplary embodiment, a hinge mechanism (200) of the present invention may comprise the front hinge element (212) in communication with the back hinge element (222). In an embodiment of a hinge mechanism (200), for example, a front arm hinge element (212) may lock with a back arm hinge element (222). Accordingly, a syringe-support apparatus of the present invention may comprise a hinge mechanism, which may allow for communication between a front arm and a back arm.

In an exemplary embodiment, a hinge mechanism (200) of the present invention may enable movement between a front arm (210) and a back arm (220) with a known scissor grip. In an embodiment, for example, the front arm (210) may further comprise a finger grip (240) and the back arm may further comprise a thumb grip (250). In an exemplary embodiment, a user may place the user's fingers in a finger grip (240) and the user's thumb in a thumb grip (250). In an embodiment, moving the finger grip (240) away from or toward the thumb grip (250) may, in view of a hinge mechanism (200), move a front arm (210) away from or toward a back arm (220). Accordingly, syringe-support mechanism of the present invention may leverage a hinge mechanism to move a front arm and a back arm relative to each other.

In an exemplary embodiment, a syringe-support apparatus (100) of the present invention may allow a user to maintain hand positioning between syringe functions and, thus, may address efficiency losses due to changing hand positioning between aspiration and injection. With reference to the syringe-support mechanism (100) described in FIG. 1, scissor-grip movement of the front arm (110) to or from the back arm (120) may enable a plunger (102) to move forward or backward through a barrel (104), respectively. As described with reference to FIG. 1, in an exemplary embodiment, because movement of the plunger (102) may couple to movement of the back arm (120), movement of the plunger (102) may couple to movement of a thumb grip (150). As described in FIGS. 1 and 2, a syringe-support apparatus (100) of the present invention may comprise a hinge mechanism (200). In an exemplary embodiment, a user may engage the hinge mechanism (200) and may leverage a full-fingered, scissor grip, rather than a two-fingered grip, to move a syringe plunger horizontally through a syringe barrel. In this way, a user may perform both syringe functions, aspiration and injection, without a change in hand position. For example, a user may simply contract or expand the user's thumb and four fingers to move the syringe plunger. That is, in an exemplary embodiment, a user may use multiple forearm flexor muscles by using multiple fingers. In this way, for example, in an embodiment, a user may increase strength and ease of injection. Accordingly, a syringe-support apparatus of the present invention may leverage a scissor-like motion to move a syringe plunger.

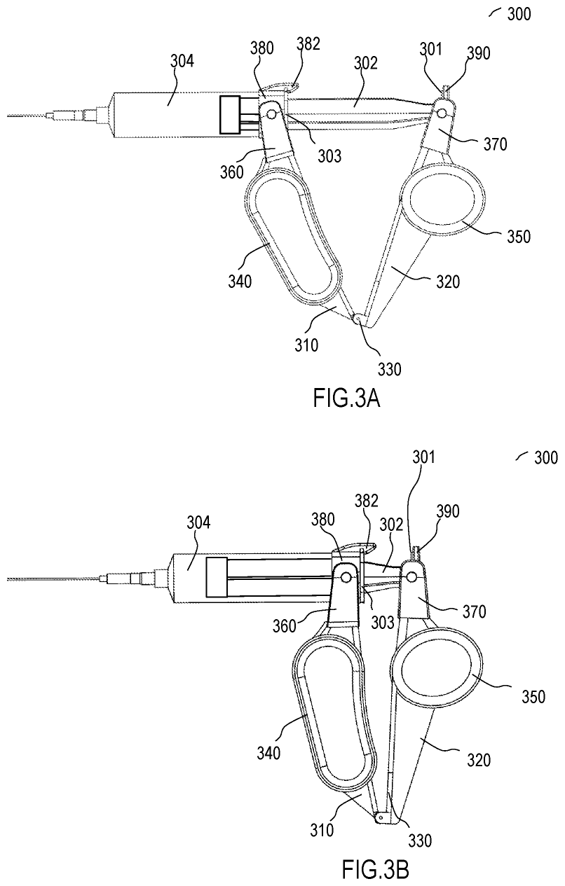

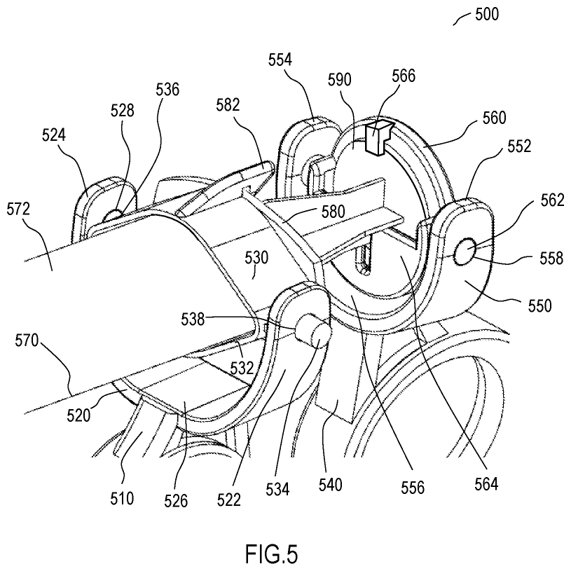

FIGS. 3A and 3B illustrate a side view of an exemplary embodiment of a syringe-support apparatus (300) with open arms and closed arms, respectively, in accordance with the present disclosure. For example, a syringe may comprise a plunger flange (301), a plunger (302), a barrel flange (303), and a barrel (304). In an embodiment, for example, syringe-support apparatus (300) may comprise a front arm (310) and a back arm (320). In an exemplary embodiment, and as described herein above in FIG. 2, a front arm (310) and a back arm (320) may pivotally communicate and lock together at a hinge mechanism (330). In an embodiment, for example, the front arm (310) may comprise a finger grip (340), a front mount (360) and a barrel support (380). As described herein below in FIGS. 5, 6 and 7, the barrel support (380) may comprise a barrel flange hook or hook mechanism (382) in communication with a barrel flange (303) to secure the syringe barrel (304) to the barrel support (380). In this way, in an exemplary embodiment, a combination of the barrel support (380) and hook mechanism (382) may secure a syringe barrel (304) in place and limit movement of the syringe barrel (304) while a syringe plunger (302) may traverse within and along the syringe barrel (304). Accordingly, a syringe-support apparatus of the present invention may secure a syringe barrel to a front arm, which may support free movement of a syringe plunger.

In an exemplary embodiment, the back arm (320) may comprise a thumb grip (350), a back mount (370), and a plunger flange support (390). A syringe-support apparatus (300) may secure a plunger flange (301) in the plunger flange support (390) and may secure a barrel flange (303) and barrel (304) in the barrel support (380). In an exemplary embodiment, the plunger flange support (390) may be a rotatable plunger flange support. In an exemplary embodiment, the plunger flange (301) may rotate on an axis as a plunger (302) may traverse an inside of a barrel (304). To that end, the plunger flange support (390) may rotate on an axis while securing the plunger flange (301). Accordingly, a syringe-support apparatus of the present invention may secure a user's fingers and thumb, as well as a syringe.

Further, a syringe-support apparatus (300) of the present invention may allow a user to perform both syringe functions, aspiration and injection, without changing hand position. As shown in FIGS. 3A and 3B, a user may place the user's fingers in the front grip (340) and the user's thumb in the thumb grip (350). With reference to FIG. 3A, using the hinge mechanism (330), a user may separate the user's fingers from the user's thumb and, thus, separate the front arm (310) from the back arm (320). Through this movement of the front arm (310) relative to the back arm (320), a syringe-support apparatus (300) may move a secured plunger flange (301) away from a secured barrel flange (303) and secured barrel (304). Accordingly, a syringe-support apparatus of the present invention may leverage scissor-like motion to aspirate fluid into syringe barrel (304).

With reference to FIG. 3B, using the hinge mechanism (330), a user may bring together the user's fingers and the user's thumb and, thus, bring together the front arm (310) and the back arm (320). Through this movement of the front arm (310) relative to the back arm (320), a syringe-support apparatus (300) may move a secured plunger flange (301) toward a secured barrel flange (303) and secured barrel (304). In other words, a user may push fluid from a secured barrel (304) for an injection by bringing together a front arm (310) and a back arm (320). In this way, in an exemplary embodiment of a syringe-support apparatus (300) of the present invention, a user may move medicine to a needle for injection and may aspirate medicine into a syringe barrel using the same hand grip, but with a different motion. Accordingly, a syringe-support apparatus of the present invention may support both aspiration and injection with a single hand position.

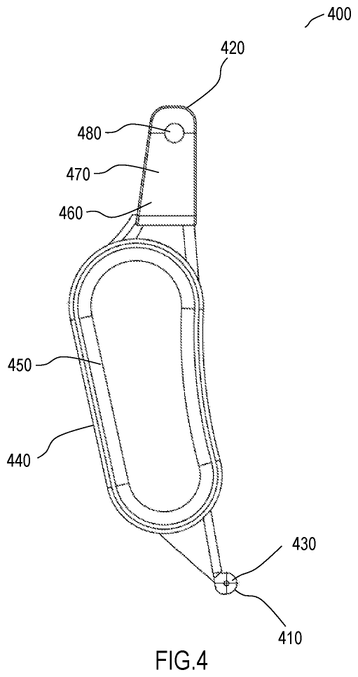

In an exemplary embodiment, a syringe-support apparatus (100), as described in FIG. 1, may control user hand movements, and thus, syringe plunger movements. For example, as described in FIGS. 3A and 3B, a user may place the user's fingers in the front grip (340) of a front arm (310). FIG. 4, illustrates a side view of an exemplary embodiment of a front arm (400) of a syringe-support apparatus in accordance with the present disclosure. In an exemplary embodiment, the front arm (400) may comprise a first end (410) and a second end (420). In an embodiment of a front arm (400), for example, a front hinge element (430) may sit close to or at the first end (410). In an exemplary embodiment, a front arm (400) may further comprise a finger grip (440) with a finger grip shape (450). In an embodiment of a front arm (400) of a syringe-support apparatus, the finger grip shape (450) may enclose and vertically position a user's pointer finger, middle finger, ring finger, and pinky finger. Further, in an embodiment, for example, the finger grip shape (450) may position the user's pointer and middle fingers more extended and less flexed than the user's ring finger and pinky finger. In an exemplary embodiment, the finger grip (440) may be slightly back tilted. In this way, for example, an embodiment of front arm (400) of a syringe-support apparatus may encourage natural finger positioning by tilting the finger grip (440). In an embodiment, for example, a user's pointer finger and middle finger may extend more and flex less than a user's ring and pinky fingers. With reference to FIG. 1, in an exemplary embodiment, a syringe-support apparatus (100) may leverage power of the pinky finger and ring finger for strength and leverage power of the pointer fingers and middle fingers for finesse. Accordingly, a syringe-support apparatus of the present invention may enclose a user's fingers.

In an exemplary embodiment of a front arm (400) of a syringe-support apparatus of the present invention, the front arm (400) may further comprise a front mount (460) at the second end (420). In an exemplary embodiment, the front mount (460) may comprise a U-shape or conform, generally, to a syringe barrel shape. The front mount (460) may secure a barrel support, which may secure a syringe barrel, as described herein below in FIG. 5. In an embodiment, for example, the front mount (460) may comprise a first side (470) of the U-Shape, as shown in FIG. 2, and a first opening (480) positioned on the first side (470). Although not shown, the front mount (460) may further comprise a second side of the U-Shape and a second opening across from the first opening (480) positioned on the first side (470). In an exemplary embodiment, as described below in FIG. 5, a front mount (460) may secure tabs of a barrel support into the first and second openings. Accordingly, a syringe-support apparatus of the present invention may comprise a mount to secure a syringe barrel.

In an exemplary embodiment of a front arm (400) of a syringe-support mechanism, the relative positions of the front grip (440) and the front mount (460) may provide injection control to a user. For example, as described in FIG. 1, a syringe may comprise, at least, a barrel flange (103), a barrel (104) and a needle (108). In an exemplary embodiment of a front arm (400) of a syringe-support apparatus, the position of the fingers may be relatively close to the needle (108). In an embodiment, for example, a front grip (440) may be positioned sufficiently close to a front mount (460), and thus, sufficiently close to a syringe barrel (104) and needle (108). Further, in an exemplary embodiment, a front arm (400) of a syringe-support apparatus may position three to four fingers in front of a barrel flange (103) in the finger grip (440). In an embodiment, for example, a front arm (400) of a syringe-support apparatus may put the user's hand closer, both vertically toward a front mount (460) and horizontally in front of a barrel flange (103), to a needle (108). In view of this relative closeness, a user's hand may be closer to a point of injection, so the user may increase user syringe control and injection stability. Accordingly, a syringe-support apparatus of the present invention may position a user's fingers close to a syringe needle.

With reference to FIG. 1, in view of FIGS. 2-4, a syringe-support apparatus (100) of the present invention may address syringe user fatigue. As described herein above, a user may generally hold a syringe with one hand. For example, a user may use two fingers to steady a barrel (104) of a syringe by gripping a barrel flange (103) with two fingers. By engaging the user's thumb, the user may push the plunger flange (101) in, move a plunger (102) horizontally along and within the barrel (104) and inject medicine via a needle (108) or through an opening into a patient. An injection, for example, may require a fair amount of force due to the volume and viscosity of the injected medicine. Users may experience fatigue in their hands, specifically in their thumbs and the basal joint from the repetitive motion and pressure of syringe injection. Accordingly, a user may experience, at least, hand fatigue by using one or two fingers to move a syringe plunger through a syringe barrel.

With reference to FIG. 1, a syringe-support apparatus (100) of the present invention may reduce user hand fatigue and increase syringe control by engaging more user muscles. More specifically, in an embodiment of a syringe-support apparatus (100), for example, a user may engage all user fingers and user thumb of one hand holding the syringe-support apparatus (100). As described herein above in FIGS. 1, 3A and 3B, a syringe-support apparatus (100) may comprise a front arm (110) comprising a finger grip (140), which may be connected via a hinge mechanism (130) to a back arm (120) comprising a thumb grip (150). In this way, a syringe-support apparatus (100) may leverage a user's hand and arm muscles. As described in FIG. 4, a finger grip shape (450) may position the user's pointer and middle fingers more extended and less flexed than the user's ring finger and pinky finger. Also, in an exemplary embodiment, the finger grip (440) may be slightly back tilted. In this way, in an exemplary embodiment, a syringe-support apparatus (100) may reflect natural, neutral wrist positioning for a user's engaged hand. An exemplary embodiment of a syringe-support apparatus (100) may, for example, position a user's wrist angled 15-20 degrees back relative to a long axis of a radius bone on the same arm. Accordingly, a syringe-support apparatus of the present invention may encourage natural, neutral finger and wrist positioning.

In an exemplary embodiment, a syringe-support apparatus (100) may leverage a user's flexor muscle mass, and the power of a user's forearm muscles, rather than merely thumb strength, to decrease hand fatigue and injection inaccuracy. Specifically, for example, by virtue of the front grip (140), thumb grip (150), and hinge mechanism (130), in an embodiment, a user may tap into a robust network of muscles by using a contracting motion of the user's entire hand rather than just using the power of the fingers (namely the thumb) to inject. Since a user may use more of the user's hand in an exemplary embodiment of the syringe-support apparatus (100) of the present invention, the user may be less likely to experience hand fatigue, or more specifically, thumb fatigue, and the user's grip is less likely to slip while injecting a syringe needle into a patient. Accordingly, a syringe-support apparatus of the present invention may leverage a user's arm muscles for power behind delivering an injection.

In an exemplary embodiment, a syringe-support apparatus (100), as described in FIG. 1, may secure a syringe to the syringe-support apparatus (100) by means of a barrel support (180) and plunger flange support (190). FIG. 5 illustrates an exploded, top perspective view of an exemplary embodiment of syringe securing mechanism (500) in accordance with the present disclosure. In an exemplary embodiment, a syringe securing mechanism (500) may comprise a front arm (510), a front mount (520) and barrel support (530). In an exemplary embodiment, a syringe securing mechanism (500) may further comprise a back arm (540), a back mount (550) and a plunger flange support (560). In an exemplary embodiment, the barrel support (530) may encase a syringe barrel (570). For example, in an embodiment, an inner surface (532) of a barrel support (530) may communicate with an outer surface (572) of a syringe barrel (570). In an exemplary embodiment, a syringe barrel (570) may be positioned centrally within a barrel support (530). In an exemplary embodiment, a barrel support (530) may communicate with and secure a barrel flange (580). For example, in an embodiment, a barrel support (530) may comprise a barrel flange hook (582), which may secure the barrel flange (580). Accordingly, a syringe-support apparatus may comprise a securing mechanism to secure a syringe to the apparatus.

In an exemplary embodiment of syringe securing mechanism (500) of the present invention, a barrel support (530) may adjust to a syringe barrel size. For example, a barrel support (530) may comprise flexible material and stretch or contract to accommodate a size of a syringe barrel (570). In an exemplary embodiment, at least a portion of the inner surface (532) of the barrel support (530) may communicate with at least a portion of an outer surface (572) of a syringe barrel (570). In an alternative embodiment, a pre-sized barrel support (530) may accommodate a size of a syringe barrel (570). In another embodiment, for example, a combination of material and size of a barrel support (530) may be employed to accommodate a size of a syringe barrel (570). For example, in an embodiment, a barrel support (530) of the present invention may be sufficiently sized or may comprise sufficiently flexible material, which may secure syringe sizes 10-50 cc. In an exemplary embodiment, a syringe securing mechanism (500) may allow a user to use a larger volume syringe, rather than having to stop and reload smaller syringes, and the injection will be easier to apply. Accordingly, a syringe-support apparatus of the present invention may secure at least one size of syringe barrel.

In an exemplary embodiment, a syringe securing mechanism (500) may comprise a front mount (520), which may comprise a first arm (522) and a second arm (524). In an exemplary embodiment, as described above, a front mount (520) may form a U-shape. For example, in an embodiment, the first arm (522) may be positioned parallel and opposite from the second arm (524) across a base (526) of the front mount (520) to form the U-shape. In an exemplary embodiment, a barrel support (530) may comprise a first tab (534) and a second tab (536), which may secure the barrel support (530) to the front mount (520). In an exemplary embodiment, the front mount (520) may secure the barrel support (530) using the first tab (534) in communication with a first opening (538) located on the first arm (522) and using the second tab (536) in communication with a second opening (528) located on the second arm (524) of the front mount (520). Accordingly, a syringe-support apparatus of the present invention may secure a barrel support to a front mount of a front arm.

In an exemplary embodiment, a syringe securing mechanism (500) may comprise a back mount (550), which may comprise a first arm (552) and a second arm (554). In an exemplary embodiment, as described above, a back mount (550) may form a U-shape. For example, in an embodiment, the first arm (552) may be positioned parallel and opposite from the second arm (554) across a base (556) of the back mount (550) to form the U-shape. In an exemplary embodiment, a back mount (550) may secure a plunger flange support (560). In an embodiment, for example, a plunger flange support (560) may comprise a first tab (562) and a second tab (not shown), which may secure the plunger flange support (560) to the back mount (550). In an exemplary embodiment, the back mount (550) may secure the plunger flange support (560) using the first tab (562) in communication with a first opening (558) located on the first arm (552) and using the second tab (not shown) in communication with a second opening (not shown) located on the second arm (554) of the back mount (550). In an exemplary embodiment, a plunger flange support (560) may further comprise a pocket (564) to receive and secure a plunger flange (590). In an exemplary embodiment, a plunger flange support (560) may further comprise a releasable hook mechanism (566), such as a hook, to further secure the plunger flange (590). In an exemplary embodiment, a plunger flange support (560) may receive and secure a plunger flange (590) with a pocket (564) and hook mechanism (566), and may release the plunger flange (590) from the pocket (564) and hook mechanism (566). Accordingly, a syringe-support apparatus of the present invention may secure a plunger flange to a back mount of a back arm.

In an exemplary embodiment of a syringe securing mechanism (500), a user may contract a front arm (510) to a back arm (520) to move a plunger flange (590) forward. In an exemplary embodiment, as the plunger flange (590) moves forward, the plunger flange support (560) may adjust to maintain constant pressure on the plunger flange (590). In an exemplary embodiment, the plunger flange support (560) may rotate via the first tab (562) in the first opening (558) and the second tab (not shown) in a second opening (not shown). That is, in an exemplary embodiment, a user may control the vertical movement of the syringe plunger as the syringe plunger travels through the syringe barrel (570) by securing the syringe plunger flange (590) in a rotatable plunger flange support (560). With reference to FIG. 1, in an embodiment, for example, a syringe-support apparatus (100) comprising an adjusting and rotating plunger flange support (190) may enable a user to apply an almost constant force throughout an injection or aspiration and to control injection speed. Accordingly, a syringe-support apparatus of the present invention may secure a plunger flange to a back mount of a back arm.

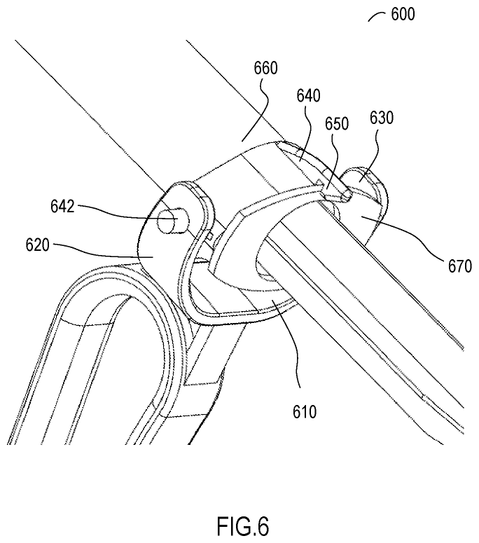

As described in FIG. 1, a syringe-support mechanism (100) of the present invention may provide for easy loading of a syringe and for easy release of the syringe. As described herein above in FIGS. 1 and 5, a syringe-support apparatus (100) of the present invention may secure a syringe in the syringe-support apparatus (100). A syringe-support apparatus (100) of the present invention may also enable removal of the syringe from the syringe-support apparatus (100). For example, a user may hold a syringe-support apparatus (100) of the present invention and separate a front arm (110) from a back arm (120). In doing so, the syringe-support apparatus (100) may provide space between a front mount (160) and a back mount (170) to remove the syringe. For example, in an embodiment, the front arm (110) and the back arm (120) of a syringe-support apparatus (100) may separate for removal of a barrel support (180) from the front mount (160) and a plunger flange (101) from the back mount (170). FIG. 6 illustrates an exploded, top, back perspective view of an exemplary embodiment of a hollow-shaped barrel flange securing mechanism (600) in accordance with the present disclosure. In an embodiment, for example, a barrel flange securing mechanism (600) may secure and stabilize, as well as release a syringe positioned within a syringe-support apparatus (100), as described in FIG. 1. In an exemplary embodiment, a barrel flange securing mechanism (600) may comprise a front mount (610), and the front mount (610) may comprise a first arm (620) and a second arm (630). In an exemplary embodiment, a barrel flange securing mechanism (600) may comprise a barrel support (640), and the barrel support (640) may comprise a first tab (642) located on a first arm (610), second tab (not shown) located on a second arm (620), and a barrel flange hook (650), which may secure a barrel flange (670). In an exemplary embodiment, a barrel support (640) may conform to at least a portion of a shape of a syringe barrel (660), for example, by receiving the syringe barrel (660) and collapsing onto an outer surface of the syringe barrel (660). As described herein above in FIG. 5, in an exemplary embodiment, the front mount (520) may secure the barrel support (530) using the first tab (534) in communication with a first opening (526) located on the first arm (522) and using the second tab (536) in communication with a second opening (528) located on the second arm (524) of the front mount (520). Accordingly, a syringe-support mechanism of the present invention may secure a barrel support to a front mount.

In an exemplary embodiment of a barrel flange securing mechanism (600), both the front mount (610) and the barrel support (640) may comprise flexible material. With reference to FIGS. 1 and 5, for example, in an embodiment, a user may compress the flexible material to receive a syringe barrel (104) or to remove a syringe barrel (104) from a syringe-support apparatus (100). With reference to FIG. 5, in an exemplary embodiment, a barrel support (530) and a barrel flange hook (582) may secure a syringe barrel to a syringe-support apparatus (100). In an exemplary embodiment, a first arm (620), a second arm (630), a barrel support (640), and a barrel flange hook (650), or any combination thereof, may comprise flexible material. For example, in an embodiment of a barrel flange securing mechanism (600), a user may compress both a first arm (620) and a second arm (630) of the front mount (610), and may move the first arm (620) and second arm (630) toward each other. In an embodiment, for example, compressing the first arm (620) and the second arm (630) may create space around a secured barrel flange (670) and release the barrel flange (670) from the barrel support (640) and a barrel flange hook (650). Accordingly, a syringe-support apparatus of the present invention may release a secured syringe by compressing a barrel securing mechanism.

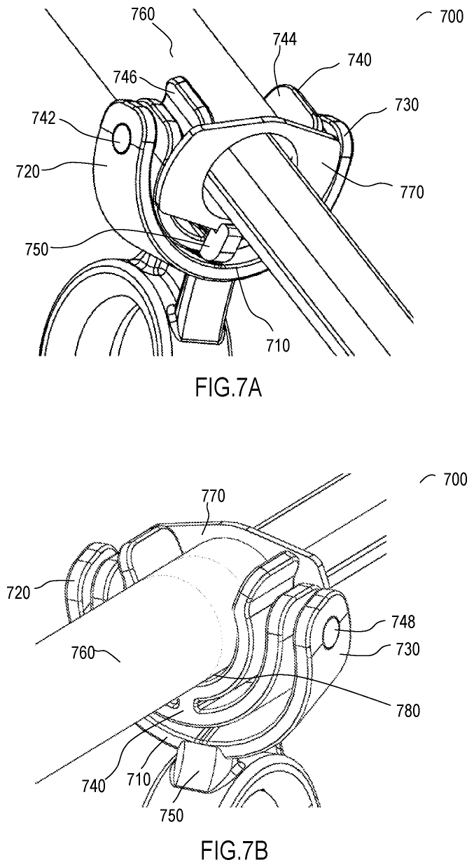

FIG. 7A illustrates an exploded, top-back perspective view of an exemplary embodiment of a saddle-shaped barrel flange securing mechanism (700) in accordance with the present disclosure. As described herein above in FIG. 6, a barrel flange securing mechanism (700) may comprise a front mount (710), and the front mount (710) may comprise a first arm (720) and a second arm (730). In an exemplary embodiment, a barrel flange securing mechanism (700) may comprise a barrel support (740), and the barrel support (740) may comprise a first tab (742) located on a first arm (720), second tab (not shown) located on a second arm (730), and a barrel flange hook (750), which may secure a barrel flange (770). In an exemplary embodiment, a barrel support (740) may stretch to accommodate at least a portion of a shape of a syringe barrel (760). In an embodiment, for example, a barrel support (740) may secure a barrel (760) by separating a first hand (744) from a second hand (746), receiving the syringe barrel (760) into a base of the barrel support (740), releasing the separated first hand (744) from the second hand (746), and enclosing the barrel (760) within the base of the barrel support (740). Alternatively, in an exemplary embodiment, a saddle-shaped barrel flange securing mechanism (700) may release a secured barrel (760) by separate a first hand (744) from a second hand (760) and release the barrel (760) from barrel support (740). Accordingly, a syringe-support apparatus of the present invention may secure and release a syringe barrel.

FIG. 7B illustrates an exploded, back-front perspective view of an exemplary embodiment of a saddle-shaped barrel flange securing mechanism (700) in accordance with the present invention. As described herein above in FIGS. 6 and 7A, a barrel flange securing mechanism (700) may comprise a front mount (710), and the front mount (710) may comprise a first arm (720) and a second arm (730). In an exemplary embodiment, a barrel flange securing mechanism (700) may comprise a barrel support (740), and the barrel support (740) may comprise a first tab (not shown) located on a first arm (710), second tab (748) located on a second arm (720), and a barrel flange hook (750), which may secure a barrel flange (770). In an exemplary embodiment, a barrel support (740) may stretch to accommodate at least a portion of a shape of a syringe barrel (760). In an embodiment, for example, a barrel support (740) may secure a barrel (760) by separating a first hand (744) from a second hand (746), receiving the syringe barrel (760) into a base (780) of the barrel support (740), releasing the separated first hand (744) from the second hand (746), and enclosing the barrel (760) within the base (780) of the barrel support (740). Alternatively, in an exemplary embodiment, a saddle-shaped barrel flange securing mechanism (700) may release a secured barrel (760) by separate a first hand (744) from a second hand (760) and release the barrel (760) from the barrel support (740). Accordingly, a syringe-support apparatus of the present invention may secure and release a syringe barrel.

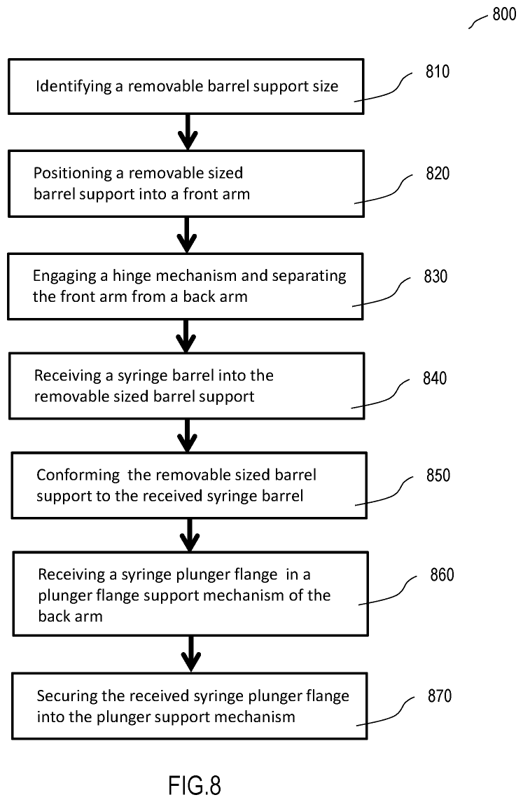

As described herein above in FIG. 1, a syringe-support mechanism (100) in accordance with the present invention may comprise a front arm (110), a back arm (120), and a hinge mechanism (130). A front arm (110) may comprise a finger grip (140) and a front mount (160), which may comprise a barrel support (180). In an exemplary embodiment, a back arm (120) may comprise a thumb grip (150) and a back mount (170), which may comprise a plunger flange support (190). FIG. 8 illustrates a flow diagram showing seating a syringe in a syringe-support apparatus (800) in accordance with the present invention. As described herein above in FIG. 5, an exemplary embodiment of syringe securing mechanism (500) may provide for securing differing sizes of syringe barrels. In an exemplary embodiment, and as described herein below, seating a syringe in a syringe-support apparatus (800) may comprise receiving a syringe barrel comprising a syringe barrel flange by a removable barrel support and receiving a syringe plunger flange by a rotatable plunger flange support. In an exemplary embodiment, seating a syringe in a syringe-support apparatus (800) may comprise identifying a barrel support size (810) and positioning a sized barrel support in a front mount (820). Accordingly, seating a syringe in a syringe-support apparatus of the present invention may comprise sizing a syringe-support apparatus to fit a barrel size.

As described in FIG. 1, a front arm (110) may comprise a front mount (160) and a barrel support (180). In an exemplary embodiment, seating a syringe in a syringe-support apparatus (800) may further comprise securing a syringe barrel in a barrel support. In an embodiment, for example, seating a syringe in a syringe-support apparatus (800) may further comprise engaging a hinge mechanism and separating the front arm from the back arm (830). In an exemplary embodiment, seating syringe-support apparatus (800) may further compromise receiving a syringe barrel in the sized barrel support (840) and contracting the sized barrel support onto the received syringe barrel (850). As described herein above, with respect to FIG. 6, at least a barrel support (640) may comprise flexible material. In an exemplary embodiment, the flexible material may adjust to receive a syringe barrel. In an embodiment, for example, receiving a syringe barrel in a barrel support (840) and conforming the barrel support to the received syringe barrel (850) may further comprise centering the received syringe barrel in the barrel support. As described in FIGS. 6, 7A, and 7B, conforming the barrel support to the received syringe barrel (850) may comprise, for example, securing the syringe barrel to the barrel support. For example, in an embodiment, conforming the barrel support to the received syringe barrel (850) may comprise snapping or collapsing a barrel support inner surface onto an outer surface of a received syringe barrel, or may comprise, for example, separating and then releasing a first hand and second hand of a barrel-shaped barrel support to secure a received syringe barrel into the barrel support. In an exemplary embodiment, conforming the barrel support may further comprise using a hook mechanism to secure a barrel flange to the barrel support. Accordingly, seating a syringe in a syringe-support apparatus of the present invention may comprise adjusting a barrel support in communication with a received syringe barrel to secure the received syringe barrel.

As described in FIG. 1, a back arm (120) may comprise a back mount (170) and a plunger flange support (190). As described in FIG. 5, in an exemplary embodiment, a plunger flange support (560) may comprise a mechanism, such as a pocket (564) for receiving a syringe plunger flange (590). In an exemplary embodiment, seating a syringe in a syringe-support apparatus (800) may further comprise receiving a syringe plunger flange in a plunger flange support mechanism (860). In an exemplary embodiment, seating a syringe in a syringe-support apparatus (800) may further comprise securing the received syringe plunger flange to the plunger flange support mechanism (870). Accordingly, a method of seating a syringe in a syringe-support apparatus of the present invention may comprise receiving and securing a syringe barrel and receiving and securing a syringe plunger flange.

It is to be understood that the various embodiments shown and described herein are to be taken as exemplary. Elements and materials, and arrangements of those elements and materials, may be substituted for those illustrated and described herein, parts may be reversed, and certain features of the present disclosure may be utilized independently, as would be apparent to one skilled in the art after having the benefit of the description herein. Changes may be made in the elements described herein without departing from the spirit and scope of the present disclosure and following claims, including their equivalents.

It is to be understood that the particular embodiments set forth herein are non-limiting, and modifications to structure, dimensions, materials, and methodologies may be made without departing from the scope of the present disclosure.

It is to be further understood that this description's terminology is not intended to limit the invention. For example, spatially relative terms, such as “front,” “back,” “top,” “bottom,” “side,” and the like, may be used to describe one element's or feature's relationship to another element or feature as intended to connote the orientation of, for example, the syringe-support apparatus as illustrated in the figures.

For the purposes of this specification and appended claims, unless otherwise indicated, all numbers expressing quantities, percentages or proportions, and other numerical values used in the specification and claims, are to be understood as being modified in all instance by the term “about” if they are not already. That is, unless indicated to the contrary, the numerical parameters set forth in the specification and claims are approximations that may vary depending on the desired properties sought to be obtained by the present disclosure.

What is claimed is:

1. An apparatus, the apparatus comprising:
a front arm comprising a first end and a finger grip loop;
a front mount in communication with the front arm at the first end;
a back arm comprising a first end and a thumb grip loop;
a back mount in communication with the back arm at the first end;
a second end of the front arm in pivotal communication with a second end of the back arm at a hinge mechanism.

2. The apparatus of claim 1, wherein the hinge mechanism engages to move the front arm relative to the back arm.

3. The apparatus of claim 1, further comprising a removable barrel support in communication with the front mount.

4. The apparatus of claim 3, the removable barrel support comprising a barrel flange hook.

5. The apparatus of claim 3, the removable barrel support comprising flexible material, wherein the flexible material at least in part conforms to an outer surface of a syringe barrel.

6. The apparatus of claim 5, the removable barrel support further comprising a saddle shape.

7. The apparatus of claim 1, further comprising a rotatable plunger flange support in communication with the back mount.

8. The apparatus of claim 7, the rotatable plunger flange support comprising a plunger flange pocket.

9. The apparatus of claim 8, the rotatable plunger flange support further comprising a plunger flange hook.

10. A method, the method comprising:
receiving a syringe barrel comprising a syringe barrel flange by a removable barrel support; and
receiving a syringe plunger flange in communication with a syringe plunger by a rotatable plunger flange support.

11. The method of claim 10, further comprising engaging a barrel flange hook mechanism in communication with the removable barrel support on the syringe barrel flange.

12. The method of claim 10, further comprising engaging a plunger flange hook mechanism in communication with the rotatable plunger flange support on the received syringe plunger flange.

13. The method of claim 10, further comprising securing the received syringe plunger flange in a pocket in communication with the rotatable plunger flange support.

14. The method of claim 10, further comprising identifying a removable barrel support size to accommodate a syringe barrel size.

15. The method of claim 10, further comprising conforming a portion of an inner surface of the removable barrel support to a portion of an outer surface of the syringe barrel.

16. A method, the method comprising:
pivotally associating a first end of a front arm with a first end of a back arm;
securing a syringe barrel flange in communication with a syringe barrel at a second end of the front arm;
securing a syringe plunger flange in communication with a syringe plunger at a second end of the back arm;
coupling a position of a thumb grip loop in communication with the back arm to a position of the syringe plunger;
engaging the thumb grip loop; and
controlling a syringe plunger linear speed with the engaged thumb grip loop as the syringe plunger travels through the syringe barrel.

17. The method of claim 16, further comprising halting the syringe plunger linear speed with the engaged thumb grip loop.

18. The method of claim 16, further comprising restricting movement of the syringe barrel by secure the syringe barrel flange to a barrel support in communication with the second end of the front arm.

19. The method of claim 16, further comprising controlling vertical movement of the syringe plunger as the syringe plunger travels through the syringe barrel by securing the syringe plunger flange to a rotatable plunger flange support.
